(19) United States
(12) Patent Application Publication
(10) Pub. No.: US 2023/0268046 A1
(43) Pub. Date: Aug. 24, 2023

(54) Method for Determining Individual Care-Product Formulations

(71) Applicant: METRICSCOSMETICS GMBH, Berlin (DE)

(72) Inventors: Franziska LEONHARDT, Berlin (DE); Domink MICHELS, Berlin (DE)

(21) Appl. No.: 18/011,452

(22) PCT Filed: Jun. 18, 2021
(86) PCT No.: PCT/EP2021/066721

§ 371 Date: Dec. 19, 2022

(30) Foreign Application Priority Data
Jun. 19, 2020 (DE) 10 2020 116 304.5

(51) Int. Cl. G16H 20/10; G06N 3/04

(52) U.S. Cl. CPC G16H 20/10; G06N 3/0409; A61B 5/441

(57) ABSTRACT

A method for the automated determination of an individual care product formulation for a user. The method according to the invention represents a hybrid approach which initially includes background knowledge of domain experts (i.e. dermatologists) who specify an initial mapping. This is augmented by a structured analysis of user feedback, which increases accuracy.

### RELATED APPLICATIONS

This application is a U.S. national phase application, claiming priority under 35 U.S.C. § 371 to PCT application PCT/EP2021/066721, filed on Jun. 18, 2021, claiming priority to German national application 10 2020 116 304.5, filed on Jun. 19, 2020, the contents of these applications incorporated by reference as if fully set forth herein in their entireties.

### FIELD OF INVENTION

The invention relates to a method as well as a device for the automated determination of an individual care product formulation for a user.

### BACKGROUND OF THE INVENTION

Cosmetic care products are playing an ever greater role in the aesthetic well-being of the population. Consumers in the past were essentially limited to the option of choosing from existing care products in an attempt to find a care product suited to their own needs by trial and error. It is true that it has always been possible for consumers to have pharmaceutically trained persons put together individual care products. However, the formulation in this case was based on the normally rather limited personal experience of the pharmacist.

US 2006/0229912 A1 discloses an automated method in which a skin treatment programme, such as massage, is determined based on image material of a user as well as answers to questions regarding skin properties.

US 2017/0281526 A1 further discloses a generic method which generates an individual care product formulation for a user based on image material and questionnaire results.

WO 2019/148116 A1 further discloses a generic method by means of which an individual care product formulation is generated for a user, likewise on the basis of image material and questionnaire results, using a neural network.

The underlying object of the invention is to provide a method for the automated determination of an individual care product formulation for a user which provides a care product formulation that is as suitable as possible.

### BRIEF DESCRIPTION OF THE DRAWINGS

The invention is described in greater detail in the following based on a preferred example embodiment with reference to the drawings, which show:

FIG. 1: A block schematic of a device for carrying out the method,

FIG. 2: A block diagram of the method according to the invention.

### DETAILED DESCRIPTION OF THE INVENTION

The object is achieved by means of the methods according to claims 1 and 4.

An embodiment of the invention comprises:
a feature input routine for establishing individual skin features of the user;
creating a user vector based on this data;
creating, based on the user vector and by means of a multilayer neural network, a feature vector containing the properties and functionalities of the care product formulation to be determined,
wherein the neural network is formed in a first formulation cycle for the user with a learning vector set consisting of parameterized expertise,
wherein the learning vector set is adapted in further formulation cycles through the capture of changes in individual skin features after the application of a previously determined care product formulation,
creating the individual care product formulation based on the feature vector and an ingredient constraint database using a loss-function optimization method,
a feature change routine for inputting changes in skin features of the user after the application of a care product according to the care product formulation in order to adapt the learning vector set.

The method according to the invention represents a hybrid approach which initially includes background knowledge of domain experts (i.e. dermatologists) who specify an initial mapping. This is augmented by a structured analysis of user feedback, which increases accuracy. This feedback loop is designed in such a manner that it is preferably continuously possible for the user to provide feedback in two ways, both explicitly, by naming and weighting positive and negative results, as well as implicitly, for example by means of sensory input such as image material of skin areas, which image material can be comparatively analysed in an automated manner. Based on the acquired feedback, the mapping of the user vector to the corresponding feature vector is treated as a machine-learning problem: the mapping is defined by means of a multilayer neural network whose edge weights are set so as to maximize an objective functional that quantifies a maximum correspondence. According to a preferred embodiment, this functional also penalizes correspondences of user and feature vectors that have been found to be insufficiently compatible according to the information acquired through the feedback loop. This is based on the insight that user information is always subject to the individual, subjectively tinted assessment of the user so that a combination of parameterized expertise and customer feedback is always incorporated in the final weighting. The interaction between the technical expertise necessary to implement this model and the professional assessment and objectivity of the domain experts in dermatology is a particular advantage of the invention.

Through the hybrid setup consisting of user feedback and domain expertise, the role of the latter changes in successive formulation cycles. If it is initially, i.e. in the initial formulation cycle, still the experts who specify the mapping, this shifts with each subsequent formulation cycle, i.e. after the application of a care product formulation obtained in an earlier formulation cycle, in the direction of the user so that the role of the experts, from knowledge-based definers to supervisory roles, likewise shifts. It is thus possible according to the invention to work initially with relatively small data sets for the users and for the data pool and thus the model to grow organically with the number of users. This also makes it possible to be selective with respect to the quality of the data and, for example, rigorously exclude potentially inconsistent data sets.

The direct incorporation of structured user feedback into the quantification of the degree of expression of individual product features allows users to be directly involved in the formulation process and the development of user priorities to be tracked if their profile is simultaneously known.

Once the feature vector has been assigned to the user in question, the formulation of the personalized care product begins. This occurs in an automated manner through the execution of an optimization method specified for an ingredient and constraint model, wherein the method preferably comprises one or more of the following constraints: minimum dosage, maximum dosage, compatibility restrictions with other ingredients.

The active substance model systematically captures potential ingredients with their respective quantitative effectiveness in relation to specific effects. The thus captured effects correspond as a whole to the features described in the foregoing so that it is possible via optimization for the optimal combination of active substances that corresponds to the user in question to be determined algorithmically. The addition of further ingredients is necessary in order to ensure product stability or, for example, a desired consistency. The set of active substances to be covered in the model can be adjusted over time. Preferably, no substances potentially posing a health risk or containing paraffin are used so that a manageable set of substances to be captured at the outset is initially included in the model with their availability and costs for the purposes of prioritization. This model can grow organically over time through the addition of further ingredients, for example on the basis of market trends and customer requests.

It was established according to the invention that a simple optimization with the objective of a maximum overlap of the entered feature vector and the features of the formulation to be determined algorithmically does not lead to an optimal formulation due to the fact there are no corresponding constraints on the individual actions of the algorithm that ensure that this final formulation guarantees the necessary compatibility and stability criteria.

According to the invention, there thus occurs the addition of a constraint model in which precisely the set of all constraints which are to be met by the care product and which lead to valid formulations is captured. These preferably include, besides dosage restrictions, paired compatibility restrictions and the use of sufficient amounts of base combinations.

The active-ingredient and constraint models have been designed by chemists and pharmacists in close collaboration with modelling and algorithm experts.

The algorithmic solution of the search for maximum correspondence between the feature vector and the features of the formulation to be determined algorithmically for the conception of the optimal care product for the user is realized by means of an optimization method (constrained optimization), wherein the above-mentioned problem is typically ill-posed and thus needs to be regularized accordingly. Domain expertise in interaction with modelling and algorithm experts is necessary both for the conception of the active-substance and constraint model as well as for an adequate regularization.

According to an advantageous further embodiment of the invention, the feature input routine provides the user with questions with the aim of enabling the input of individual skin features of the user in order to capture a plurality of the following data points of the user: skin type, degree of sensitivity, tendency to irritation, formation of blood vessels or veins, pigment spots, redness, impurities, moisture loss, firmness, elasticity, tendency to flaky patches, wrinkles, pore appearance. According to the invention, the user vector is generated from as many of these skin features as possible. Input occurs via the user's answering questions as well as via an image evaluation using photographic images of skin areas at preferred locations.

According to an advantageous further embodiment, the feature input routine enables an input of further non-skin-related data of the user: gender, living environment, stress levels, sleep habits, diet, water consumption, smoking habits, travel habits, sports activities, UV radiation exposure, which are input via questions and answers.

According to an advantageous further embodiment, the feature input routine enables the input of target qualities of the user for the care product, preferably a care product feel, a care product colour and/or a care product fragrance. Preferably, the answers and the image evaluations are compared with one another for the feature acquisition. Photographic images are preferably produced under different illumination spectra (infrared, red, blue, UV) in order to better determine individual features.

According to an advantageous further embodiment, the learning vector set comprises feature vectors of other users, whereby the accuracy of the formulations is improved with an increasing number of users due to the experience of these users.

According to an advantageous further embodiment, the loss-function optimization method determines a global minimum by means of a gradient method or the Monte Carlo algorithm. The loss-function optimization method uses the loss function

$$L = {a}_{i} ( F {V}_{í}^{Customer} , {FV}_{i}^{Product} ) + b {\sum }_{j} ( I {V}_{j} )$$

with the target feature vector FV^{Customer} and the care product feature vector FV^{Product} as well as

$${a}_{i} = \{ \begin{matrix}1 , & F {V}_{í}^{Customer} \geq F {V}_{i}^{Product} \\ 0.4 , & F {V}_{í}^{Customer} < {FV}_{i}^{Product}\end{matrix} ,$$

$$and b = 0. 01$$

The following indicator function is used:

$$I {V}_{j} = \{ \begin{matrix}1 , & is contained in the formulation \\ 0 , & is not contained in the formulation\end{matrix}$$

The preferred Monte Carlo algorithm has the following sequence

Step n→n+1:
for a random active substance j, presence (=1) or non-presence (=0) is interchanged in the IV vector,
a loss value L₍ₙ₊₁₎ is calculated,
L₍ₙ₊₁₎ is compared with the previous value of L₍ₙ₎,
if L₍ₙ₊₁₎≤L₍ₙ₎, then the step is accepted,
if L₍ₙ₊₁₎>L₍ₙ₎, the step is accepted or rejected with the standard Boltzmann probability exp((L₍ₙ₎−L₍ₙ₊₁₎)/T).
T is a normalization parameter that is reduced at each step by a factor T₍ₙ₊₁₎=F·T₍ₙ₎ with 0<F<1. In the application according to the invention, F=0.995, for example, is stable. The algorithm converges after several hundred thousand steps.

In a gradient method, a step n→n+1 resembles the following:
For each j in IVⱼ, the presence or non-presence is interchanged based on the current IV vector.
L is calculated for each new vector IV
The change with the lowest L is accepted.

The algorithm converges after several thousand steps.

According to an embodiment of the invention, the object is achieved by a method for the automated determination of an individual care product formulation P_{User} for a user, consisting of a number of ingredients Iⱼ, in respective quantities λⱼ,
wherein the ingredients Iⱼ are determinable from a totality M (1≤j≤M) of available ingredients I_{M}, based on a target composition P_{z} for the user having a number of properties Fᵢ out of a total number of properties N, wherein each property Fᵢ is specified (1≤i≤N) with a degree αᵢ and a priority βᵢ;
wherein the degree αᵢ indicates the intensity of this property desired by the user by [0,1]⊂R;
wherein the priority βᵢ, indicates the priority for the user by [0,1]⊂R;
and a user-specific ingredient matrix I_{User} is determined as I=(Δ_{i,j})∈[−1,1]^{N×M} which indicates how the degree αᵢ of the property Fᵢ within a product composition changes by using a quantity λⱼ=1 of the ingredient Iⱼ;
wherein for each ingredient Iⱼ a specific price pⱼ per unit of weight or volume is specified and a user-specific price p_{User}=(p₁, P₂, . . . p_{M}) is determined therefrom; and the product composition P_{User} is determined using a loss-function optimization method argmin (λ₁, λ₂ . . . λ_{M}){F(λ₁, λ₂ . . . λ_{M})} with

F=γ₁Σᵢ₌₁^{N}βᵢ∥αᵢ−(λ₁Δ_{i,1}+λ₂Δ_{i,2}+ . . . +λ_{M}Δ_{i,M})∥²+γ₂Σⱼ₌₁^{M}λⱼpⱼ;

wherein γ₁, γ₂ ∈[0,1] are weights which prioritize quality and price, respectively; and P_{User}, I_{User} and p_{User} are determined subject to the following three constraints:
1: Quantity: C_{quant}={γ1+λ₂+ . . . +λₘ=λ^{max}}
2: Maximum dosage: Cₘₐₓ={λ₁≤λ₁^{max}, λ₂≤λ₂^{max}, . . . , λₘ≤λₘ^{max}}
3: Non-compatibility: C_{nc}={(k,l)∈{1,2, --,m}²|Iₖ and I₁ cannot be combined}
and outputting the determined product composition P_{User} to a care-product generation unit for generating a care product with this product composition P_{User}.

The priority βᵢ with [0,1]⊂R is specifiable in particular through prior input by the user via an input means, where appropriate while taking into account generically stored values.

The weighting of the quality occurs through the choice of a corresponding coefficient γ1. The weighting of the price occurs analogously by means of the coefficient γ2. Typically, these coefficients γ₁, γ₂∈[0,1] are to be selected in such a manner that γ1+γ2=1. An equal weighting of quality and price is realized in this case by the coefficient choice γ1=γ2=0.5.

The solution of the optimization problem corresponds to argmin (λ₁, λ₂ . . . . λₘ){F(λ₁, λ₂ . . . λ_{M})}.

With regard to Constraint 1, λ^{max} must be fixed. Here it makes sense to work with relative values and accordingly set λ^{max}=1. With regard to Constraint 2, the maximum dosage λ₁^{max}, λ₂^{max}, . . . , λₘ^{max} corresponds in this case to the relative parts of the corresponding ingredients lying in the interval [0,1] in relation to the total formulation. In practice, the values λ₁^{max} are subject to both chemical and regulatory constraints. If, for example, azelaic acid is potentially used in a formulation, it must not be overdosed: if, for example, a maximum azelaic-acid content of 25% is permitted in the total formulation, the corresponding value λ₁^{max}=0.25 must be set.

With respect to Constraint 3, non-compatibilities must accordingly be completely captured. For example, AQUAXYL™ must not be combined with salicylic acid nor with Bio-Placenta. Salicylic acid and Bio-Placenta must also not be combined. The corresponding constraint would read:
Cnc={(AQUAXYL™, salicylic acid), (AQUAXYL™, Bio-Placenta). (Bio-Placenta, salicylic acid), (salicylic acid, AQUAXYL™), (Bio-Placenta, AQUAXYL™), (salicylic acid, Bio-Placenta)}.

The determination of the global minimum occurs by means of a gradient method or a Monte Carlo method.

The output of the product composition P_{User} to a care-product generation unit can occur indirectly through the output of a corresponding data record that can be used to control the care-product generation unit, or it can occur directly through the direct initiation of the generation of the care product based on the product composition P_{User}.

The sequence of the Monte Carlo algorithm is illustrated in the following with a concrete example.

The neural network trained by means of the learning vector set first generates, from the user vector, a user-specific feature vector F_{User} which defines the features or properties of the care product formulation. This results by way of example in the following feature vector (right column). The features corresponding to the respective quantitative values of the components of the feature vector are listed in the left column.

| Feature | Feature Vector F |
|---|---|
| F₁: Protection against UV radiation | 0.0 |
| F₂: Prevention of moisture loss | 0.6 |
| F₃: Elimination of dry patches | 0.6 |
| F₄: Elimination of oily t-zone | 0.0 |
| F₅: Matting effect | 0.0 |
| F₆: Suitability for sensitive skin | 0.4 |
| F₇: Elimination of impurities | 0.4 |
| F₈: Elimination of redness | 0.6 |
| F₉: Protection against urban smog | 0.4 |
| F₁₀: Treatment of hyperpigmentation | 0.0 |
| F₁₁: Wrinkle reduction | 0.0 |
| F₁₂: Refinement of pores | 0.0 |
| F₁₃: Treatment of lacklustre skin | 0.0 |
| F₁₄: Protection against blue light | 0.4 |
| F₁₅: Treatment of redness after shaving | 0.6 |
| F₁₆: Treatment of smoker skin | 0.0 |
| F₁₇: Anti-aging effect | 0.4 |
| F₁₈: Suitable for sleep deprivation | 0.0 |

The individual components of the feature vector are always real numbers in the closed interval [0,1], wherein 0 is to be equated with no expression of the feature in question and 1 with maximum expression.

The following active substance model is also given by way of example to demonstrate the algorithmic procedure.

This reduced active substance model comprises by way of example exclusively the ingredient Arctalis™, the effect of which is quantified here analogously to the feature vector for the respective features. Its components are “Aqua, Propanediol, Xanthan Gum, Glyceryl Caprylate, Pseudoalteromonas Ferment Extract”.

The elements of this quantification are always real numbers in the closed interval [−1,1], wherein 0 is to be equated with no effectiveness of the feature in question and 1 with maximum effectiveness. In contrast to the feature vector, negative values are also explicitly permitted here if the ingredient influences the respective effect negatively. This can be seen by way of example in the quantification of the ingredient Arctalis™.

| Arctalis effect | Ingredient Vector I |
|---|---|
| I₁: Protection against UV radiation | 0.0 |
| I₁: Prevention of moisture loss | 0.8 |
| I₂: Elimination of dry patches | 0.4 |
| I₃: Elimination of oily t-zone | −0.2 |
| I₄: Matting effect | −0.2 |
| I₅: Suitability for sensitive skin | 0.4 |
| I₆: Elimination of impurities | −0.2 |
| I₇: Elimination of redness | 0.0 |
| I₈: Protection against urban smog | 0.0 |
| I₉: Treatment of hyperpigmentation | 0.0 |
| I₁₀: Wrinkle reduction | 0.6 |
| I₁₁: Refinement of pores | −0.2 |
| I₁₂: Treatment of lacklustre skin | 0.0 |
| I₁₃: Protection against blue light | 0.2 |
| I₁₄: Treatment of redness after shaving | 0.2 |
| I₁₅: Treatment of smoker skin | 0.0 |
| I₁₆: Anti-aging effect | 0.6 |
| I₁₇: Suitable for sleep deprivation | 0.0 |

Further active substances (for example brand names “B-Circadin”, “Neurophroline” or “HySilk”) must be captured in the active substance model analogously as well as the chemical complexes used as bases of the care products. Furthermore, it must be specified in the constraint model which combinations of ingredients are impermissible. For example, Arctalis™ should not be combined with salicylic acid, as is captured in the following.

In the present example, the Monte Carlo algorithm already converges after around 15,000 iterations. As a result, the algorithm determines a product-based formulation for normal skin containing 4 components Arctalis™, B-Circadin®, Neurophroline™ and HySilk®.

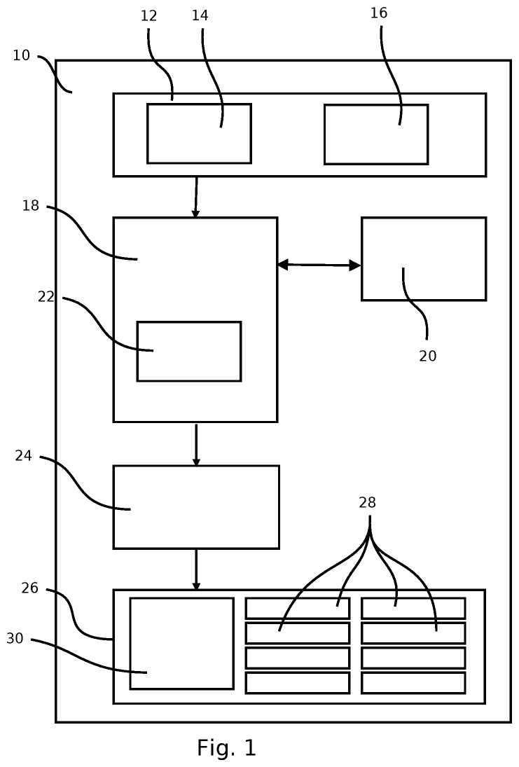

FIG. 1 shows a block schematic of a device 10 for carrying out the method, comprising a feature input unit 12 comprising an interaction unit 14 for an interactive data input by a user. The interaction unit 14 preferably comprises a screen for the visual output of questions to the user, which can be entered by the user through the operation of buttons or an interactive screen or acoustically. The feature input unit 12 further comprises a photo input unit 16 for generating photographic images of skin areas, for example of the nose, forehead or chin area of the user or the back of the hand. A data processing unit 18 evaluates the user data entered via the feature input unit 12 and generates a user vector, which is stored in a memory unit 20.

The user vector can have a large number of components that are determined by the answers to the questions put to the user. In particular:
Age,
Gender,
General feeling of skin with answer alternatives “very dry”, “dry”, “oily”, “combination skin”, “normal”,
Sensitivity of the skin with answer alternatives “insensitive”, “sensitive”, “hypersensitive”
Self-assessment of skin type with answer alternatives Types I to VI, e.g. “Skin type III tans easily in summer. The intrinsic protection time is approx. 30 min. This type rarely gets freckles, occasionally sunburns and has medium blond to brown hair.”
Feel of the skin in the morning before cleansing or application of products with answer alternatives “dry and tight”, “dry in certain zones”, “oily”, “oily only in the t-zone”, “normal”, “don't know”.
Skin irritation after shaving with answer alternatives “never”, “sometimes”, “often”, “always”.
Skin allergies with binary answer alternatives “yes” and “no”,
Vein visibility with binary answer alternatives “yes” and “no”,
Skin condition in the evening with answer alternatives “generally dry”, “partly dry”, “oily”, “normal”, “don't know”,
Pregnancy with binary answer alternatives “yes” and “no”,
Solar irradiation with subjective assessment by user 0 to 4.

In an image input routine for inputting image data of at least one skin section of the user, the user is preferably requested to take a so-called “selfie”, i.e. a photographic facial image, which is preferably evaluated and weighted (0-100%) according to the following criteria:
Skin type,
Degree of sensitivity,
Tendency to irritation,
Formation of blood vessels or veins,
Pigment spots,
Redness,
Impurities,
Moisture loss,
Firmness,
Elasticity,
Tendency to flaky patches,
Wrinkles,
Pore appearance,
Circles under eyes,
Bags under eyes,
Reflective properties of the skin.

The user vector is then created automatically based on the entered data.

The data processing unit 18 comprises a multilayer neural network 22. At least one learning vector set is stored in the memory unit 20 for the learning of the network 22. The neural network 22 trained by means of the learning vector set generates, from the user vector, a feature vector that contains the features of the care product formulation.

A learning vector set contains correlations between properties of the available substances that can be a component of the care product formulation as well as interactions with other substances. The learning vector set was generated on the basis of dermatological expertise as well as preferably on the basis of knowledge acquired from other users.

The learning vector set is further updated by experiential knowledge based on previous treatments of the user with previously determined care product formulations and the resulting effects in a feedback loop. To this end, the interaction unit 14 can output questions regarding an improvement or worsening of certain skin features to the user after a treatment with a certain care product formulation, the answers being incorporated in the learning vector set.

The data processing unit 18 is connected to a data output unit 24, which outputs the thus determined care product formulations. The data output unit 24 can comprise a visual display device. The data output unit 24 can be coupled to a care-product generation unit 26, which mixes the care product on the basis of the formulation located in the data output unit 24. To this end, the care-product generation unit 26 contains a number of containers 28 for receiving substances which are potentially or necessarily components of the care product to be generated, as well as a mixing device 30 which mixes the care product from the substances in the containers 28 based on the determined care product formulation and dispenses it into a suitable container.

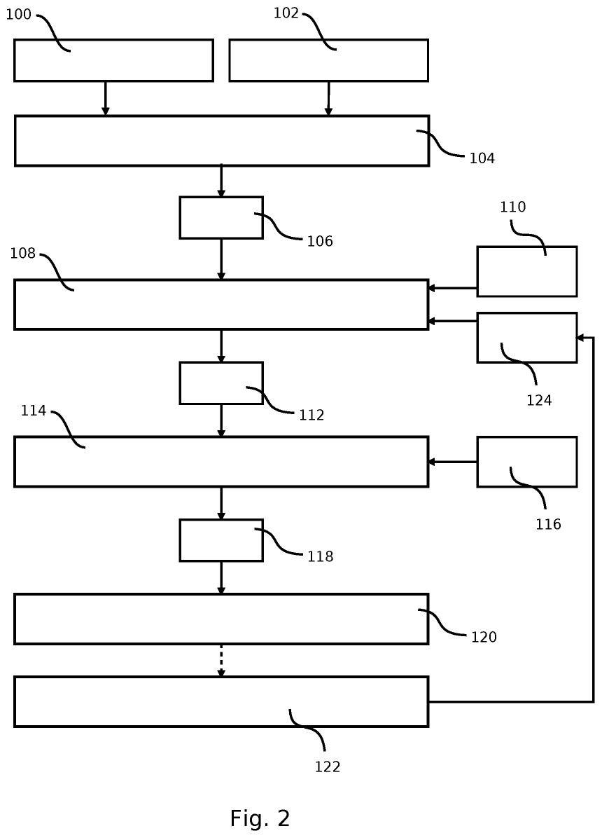

FIG. 2 illustrates the formulation process again. In an interactive data input step 100, the user enters data regarding his or her skin profile by answering questions. In an optical data input step 102, a camera is used to take a plurality of photographs of one or more skin sections. In this step 104, a data evaluation is carried out, in particular by means of data processing algorithms, in order to extract specific skin features from the photographic images. A plausibility check is also carried out with the acquired image information in the step 104 in order to check whether the skin properties entered by the user correspond to the optically detected skin properties. In the event of discrepancies, supplementary questions can be put to the user or further photographs can be taken, e.g. under other illumination angles or other illumination spectra, and evaluated. As a result, a user vector 106 is determined in step 104 that contains all user information that is more or less relevant for the formulation.

In step 108, a multilayer neural network generates, based on the user vector 106 as well as a learning vector set stored in a knowledge database 110, a feature vector 112 containing the features of the care product formulation to be determined.

In step 114, the care product formulation 118 is determined from the feature vector 112 by means of an optimization method based on an ingredient constraint database 116 and using a loss-function optimization method.

In step 120, the care product is generated from available substances based on the care product formulation 118 and filled into a suitable container for the user.

The user will subsequently apply the care product to the skin over a suitable period of time of 1-3 weeks. The user can observe the effects and then, in a step 122 that closely resembles the interactive data input step 100, enter his or her experiences and observations, which are stored in a user database 124 and which in a subsequent formulation cycle become part of the learning vector set for the learning of the neural network 22 in step 108.

1. Method for the automated determination of an individual care product formulation P_{User} for a user, consisting of a number of ingredients Iⱼ, in respective quantities λⱼ,
wherein the ingredients are determinable from a totality M (1≤j≤M) of available ingredients I_{M},
based on a target composition P_{Z} for the user having a number of properties Fᵢ out of a total number of properties N, wherein each property Fᵢ is specified (1≤i≤N) with a degree αᵢ and a priority βᵢ;
wherein the degree αᵢ indicates the intensity of this property desired by the user by [0,1]⊂R;
wherein the priority βᵢ indicates the priority for the user by [0,1]⊂R;
and a user-specific ingredient matrix I_{User} is determined as I=(Δ_{i,j})∈[−1,1]^{N×M}, which indicates how the degree αᵢ of the property Fᵢ within a product composition changes by using a quantity λⱼ=1 of the ingredient Iⱼ;
wherein for each ingredient Iⱼ a specific price pⱼ per unit of weight or volume is specified and a user-specific price p_{User}=(p₁, P₂, . . . p_{M}) is determined therefrom;
and the product composition p_{User} is determined using a loss-function optimization method argmin (λ₁, λ₂ . . . . λ_{M}){F(λ₁, λ₂ . . . λ_{M})} with
F=γ₁Σᵢ₌₁^{N}βᵢ∥αᵢ−(λ₁Δ_{i,1}+λ₂Δ_{i,2}+ . . . +λ_{M}Δ_{i,M})∥²+γ₂Σⱼ₌₁^{M}λⱼpⱼ;
wherein γ₁, γ₂∈[0,1] are weights which prioritize quality and price, respectively; and P_{User}, I_{User} and p_{User} are determined subject to the following three constraints:
1: Quantity: C_{quant}={γ1+λ₂+ . . . +λₘ=λ^{max}}
2: Maximum dosage: Cₘₐₓ={λ₁≤λ₁^{max}, λ₂≤λ₂^{max}, . . . , λₘ≤λₘ^{max}}
3: Non-compatibility: C_{nc}={(k,l)∈{1,2, --,m}²|Iₖ and I₁ cannot be combined}
and outputting the determined product composition P_{User} to a care-product generation unit for generating a care product with this product composition P_{User}.

2. The method according to claim 1, wherein the loss-function optimization method determines a global minimum by means of the Monte Carlo method.

3. The method according to claim 1, comprising a feature input routine for the user for establishing individual skin features of the user.

4. Method for the automated determination of an individual care product formulation for a user, comprising:
a feature input routine for establishing individual skin features of the user;
creating a user vector based on this data;
creating, based on the user vector and by means of a multilayer neural network, a feature vector containing the properties and functionalities of the care product formulation to be determined,
wherein the neural network is formed in a first formulation cycle for the user with a learning vector set consisting of parameterized expertise,
wherein the learning vector set is adapted in further formulation cycles through the capture of changes in individual skin features after the application of a previously determined care product formulation,
creating the individual care product formulation based on the feature vector and an ingredient constraint database using a loss-function optimization method,
a feature change routine for inputting changes in skin features of the user after the application of a care product according to the care product formulation in order to adapt the learning vector set.

5. The method according to claim 3, wherein the feature input routine provides the user with questions with the aim of enabling the input of individual skin features of the user in order to capture a plurality of the following data points of the user: skin type, degree of sensitivity, tendency to irritation, formation of blood vessels or veins, pigment spots, redness, impurities, moisture loss, firmness, elasticity, tendency to flaky patches, wrinkles, pore appearance.

6. The method according to claim 1, comprising an image input routine for the entry of image data of at least one skin section of the user.

7. The method according to claim 6, comprising an image analysis step for determining one or more of the following data points of the user based on the entered image data: skin type, degree of sensitivity, tendency to irritation, formation of blood vessels or veins, pigment spots, redness, impurities, moisture loss, firmness, elasticity, tendency to flaky patches, wrinkles, pore appearance.

8. The method according to claim 4, wherein the feature input routine comprises an input of further non-skin-related data, preferably at least one of the following data points of the user: gender, living environment, stress levels, sleep habits, diet, water consumption, smoking habits, travel habits, sports activities, UV radiation exposure.

9. The method according to claim 4, wherein the feature input routine comprises an input of care-product target qualities of the user, preferably one or more of the following target qualities: care product feel, care product colour, care product fragrance.

10. The method according to claim 4, wherein the learning vector set comprises feature vectors of other users.

11. The method according to claim 4, wherein the ingredient constraint database comprises constraints relating to the ingredients or combinations thereof, preferably one or more of the following conditions: minimum dosage, maximum dosage, compatibility restrictions with other ingredients.

12. The method according to claim 4, wherein the loss-function optimization method determines a global minimum by means of a gradient method or a Monte Carlo method.

13. Device for carrying out the method according to claim 4, comprising:
a feature input unit for an interactive data input through entry of answers to output questions as well as for taking photographic images,
a memory unit for storing the input data and at least one learning vector set,
a data processing unit which establishes a user vector based on the entered data of the user and which further determines a feature vector based on the user vector and properties and functionalities of care product components,
a multilayer neural network which generates an individual care product formulation based on the feature vector, a learning vector set and an ingredient constraint database using a loss-function optimization method, and
an output unit for outputting the care product formulation.

14. Device for carrying out the method according to claim 1, comprising
a data processing unit which generates an individual product composition P_{User} on the basis of a specified target composition P_{D} for a user having a number of properties Fᵢ out of a total number N of properties using the loss-function optimization method, and,
an output unit for outputting the care product formulation.

15. The device according to claim 13, characterized in that the device comprises a care-product generation unit, which comprises containers with potential care product ingredients, as well as a mixing unit, wherein the device generates a care product from care product components on the basis of the care product formulation.

16. The method according to claim 2, further comprising a feature input routine for the user for establishing individual skin features of the user.

17. The method according to claim 4, wherein the feature input routine provides the user with questions with the aim of enabling the input of individual skin features of the user in order to capture a plurality of the following data points of the user: skin type, degree of sensitivity, tendency to irritation, formation of blood vessels or veins, pigment spots, redness, impurities, moisture loss, firmness, elasticity, tendency to flaky patches, wrinkles, pore appearance.

18. The method according to claim 4, further comprising an image input routine for the entry of image data of at least one skin section of the user.

19. The method according to claim 18, comprising an image analysis step for determining one or more of the following data points of the user based on the entered image data: skin type, degree of sensitivity, tendency to irritation, formation of blood vessels or veins, pigment spots, redness, impurities, moisture loss, firmness, elasticity, tendency to flaky patches, wrinkles, pore appearance.

20. The device according to claim 14, characterized in that the device comprises a care-product generation unit, which comprises containers with potential care product ingredients, as well as a mixing unit, wherein the device generates a care product from care product components on the basis of the care product formulation.
